(19) United States
(12) Patent Application Publication
(10) Pub. No.: US 2006/0292196 A1
(43) Pub. Date: Dec. 28, 2006

(54) Formulation for obtaining a pharmaceutical composition, method for obtaining and use thereof

(76) Inventors: Valodia Padiurashvili, Tbilisi (GE); Tamazi Janashvili, Tbilisi (GE)

Correspondence Address: LADAS & PARRY, 26 WEST 61ST STREET, NEW YORK, NY, 10023, US

(21) Appl. No.: 11/445,918

(22) Filed: Jun. 2, 2006

Related U.S. Application Data
(63) Continuation-in-part of application No. PCT/GE04/00006, filed on Dec. 2, 2004.

(30) Foreign Application Priority Data
Dec. 3, 2003 (GE) AP 2003 005325
Nov. 26, 2004 (GE) AP 8516

(51) Int. Cl. A01N 25/00; A01N 59/00; A61K 33/00

(52) U.S. Cl. 424/405; 424/718

(57) ABSTRACT

Invention relates to the field of medicine and may be used in medicine, veterinary, biology, biotechnological, chemical-pharmaceutical and food industry, as well as for domestic purposes, etc. as a reagent providing antimicrobial, antiviral and fungicidal capability. Objective of the proposed invention is a creation of pharmaceutical composition that is innocuous in use, simple for production, cheap and has high disinfecting, sterilizing and antiseptic properties. Essence of the proposed invention is in performing mixing of ammonia synthesis catalyst waste residues and citric acid with ethyl alcohol, adding to the mixture a weak nitric acid, dilution with water, interaction of the mixture reagents till receiving a transparent fraction, purification of the transparent fraction from admixtures, adding aromatizer.

The invention refers to the formulation for obtaining a pharmaceutical composition and the method for obtaining thereof, as well as use of the pharmaceutical composition as a means for disinfection, sterilization and antiseptics for destroying pathogenic and conditioned pathogenic microorganisms, as an antiseptic for destroying or suppression of vital activity of potentially dangerous for the health of humans and animals, microorganisms in wounds, on the skin, in mucous membrane and cavities with the purpose of infection processes development prevention.

The invention may be used in medicine, veterinary, in biology, pharmaco-chemical and food industries, as well as for household appointment, etc.

As it is known, disinfectant, sterilizing and antiseptic substances are widely used at hospitals and other medicinal and prophylactic institutions for antimicrobial treatment of various surfaces. They are primarily used for prevention of infectious diseases and hospital infections.

Conventionally antimcrobial substances may be divided in three groups:
1) disinfecting and sterilizing means used for destruction of microorganisms in an environment;
2) antiseptic means used locally against microbes on the skin surfaces, mucous membrane, in wounds, and cavities;
3) chemical and therapeutic means used for treatment of infection diseases.

Generally the spectrum of effect of disinfecting means is much wider. They represent chemical agents of such a spectrum that destroy or suppress the propagation of microbes on the surface or within the live organisms or in some substances.

There are known many disinfecting means characterized with the above properties. For example, such are boric or salicylic acids and different kinds of disinfecting and antiseptic means made on their base. [Pharmaceutical and Therapeutic Reference Book, TRINUS F. P., p. 453, 477].

These acids when using may be characterized as weak disinfecting ability.

There is also known a widely spread disinfecting means “Lysoformin-3000” comprising glyoxal, glutaral and didecyldimethylammonium chloride and other components.

[Methodological Instructions of Using “Lysoform 3000”, Producer—Dr. Hans Rosemann GmbH—Germany].

The process of obtaining said disinfectant means is complex. At the same time, before its application strict safety rules should be observed that protect respiratory tracts, skin and eyes. After application of the solution it is desirable to clean everything additionally with bidistilled water.

At the same time, the disinfecting solution must be stored in the dark, air-convection vault, in the place inaccessible for general use, as it has self-inflammable and explosive features, and when used it is necessary to apply the means of the personal protection, in view of poisoning of the maintenance staff.

There is known the disinfecting means “Farmadez” comprising hydrogen peroxide, potassium fluoride with promoter added therein. [Internet: http://www.farmahim.ru, preparation “Farmadez”, 20.05.2002 (was found on Jul. 11, 2003)].

The action time of said disinfectant for achieving the final target is 60 or 120 minutes that is much greater in comparison with the other disinfectants. At the same time, the aggressive features of hydrogen peroxide and potassium fluoride should be marked and at their interaction a very aggressive and poisonous substance such as fluorine acid is produced. That is why, their safety norms and means must be observed and applied. Special conditions for their storage are also required.

There is also known a disinfecting means obtained by mixing hydrogen peroxide and formic acid at simultaneous radiation by ultraviolet source having the wave length 290-310 nm. [USSR Author's Certificate No 1172514 of May 10, 1983].

However, this method is characterized with a technological complexity conditioned by necessity performing reagents interaction process simultaneously with ultra-violet radiation which is a catalyst. In addition, ultra-violet irradiation has a negative effect on living organism. At performing said method hydrogen peroxide and formic acid are used that cause burn on the skin of a human and with this reason use of individual means of protection is necessary.

It should be also marked that application of these substances [2,3,4] as an antiseptic means is not known.

Purpose of the present invention is to create simple for manufacture, cheap and innocuous pharmaceutical composition with high disinfecting and antiseptic properties.

For achieving said purpose a formulation for obtaining a pharmaceutical composition is proposed, said pharmaceutical composition comprises a weak nitric acid, ammonia synthesis catalyst waste residues, citric acid, ethyl alcohol, water and aromatizer, under the following proportion of components, in weight %:

| | |
|---|---|
| Weak nitric acid | 0.3-10.0 |
| Ammonia synthesis catalyst waste residues | 0.1-5.0 |
| Citric acid | 0.1-2.0 |
| Ethyl alcohol | 0.1-5.0 |
| Aromatizer | 0.05-0.1 |
| Water | the rest |

At the same time, ammonia synthesis catalyst waste residues comprise not less than 50% of iron oxide, chrome oxide and graphite.

A method for obtaining the pharmaceutical composition provides for: mixing of ammonia synthesis catalyst waste residues and citric acid with ethyl alcohol till obtaining a homogeneous mass; adding weak nitric acid in the mixture; dilution with water; interaction of mixture reagents till obtaining a transparent fraction; purification of the obtained transparent fraction from admixtures; adding the aromatizer, with this, the components make in weight %:

| | |
|---|---|
| Weak nitric acid | 0.3-10.0 |
| Ammonia synthesis catalyst waste residue | 0.1-5.0 |
| Citric acid | 0.1-2.0 |
| Ethyl alcohol | 0.1-5.0 |
| Aromatizer | 0.05-0.1 |
| Water | the rest |

Interaction of reagents is carried out in a closed system, for example, a reactor or similar to the reactor technological installation at the temperature 25-90° C. and under pressure 1.013.10⁴-1.013.10⁵ Pa.

Obtained by the above method pharmaceutical composition is remarkable for high chemical resistance and stability that conditions its application as a disinfecting, sterilizing and antiseptic means, as well as a reagent providing antimicrobial, antivirus and fungicidal activity.

Application as the above described means is also performed by aqueous pharmaceutical composition with the concentration 0.025-3.0%.

Treatment of the surfaces is performed by dipping up to complete covering in the solution, washing the walls and surfaces with a stream, aerosol spraying, irrigation, cleaning of the surfaces with a rag wetted in the solution, washing, applying bandage wetted in the solution.

Proposed pharmaceutical composition is obtained as follows:

In the reactor with a mixer (depending on the total weight of the formulation) 0.1÷5.0% ammonia synthesis catalyst waste milled residues, 0.1÷2.0% citric acid are mixed with 0.1÷5.0% of 96% ethyl alcohol till obtaining of homogeneous mass. After that, to the mixture 0.3÷10.0% of 57% solution of nitric acid is added and the mixture is diluted with water to 100% and mixed again. Further the mixture is heated to temperature 25-90° C and is allowed to hold under pressure 1.013.10⁴-1.013.10⁵ Pa, the interaction of reagents is performed till obtaining a transparent fraction. Then this fraction is poured in another vessel, purified from admixtures by conventional methods and 0.05-0.1% of aromatizer, for example, essence—concentrated aqueous alcoholic solution of essential oils, is added.

At obtaining of the proposed pharmaceutical composition, use of less or more amount of the terminal value of the applied components has a negative effect on the quality or use conditions of the obtained disinfecting means.

For obtaining the pharmaceutical composition, when adding to the formulation a weak nitric acid lower than 0.3% of the total quantity of formulation, the antimicrobial and antiviral properties of the pharmaceutical composition are reduced, and when adding higher than 10.0%—it has a negative effect on the quality and properties of the pharmaceutical composition.

For obtaining the pharmaceutical composition when adding to the formulation ammonia synthesis catalyst waste residues lower than 0.1% of the total quantity of formulation, the stability of the obtained composition and efficiency of its use is reduced, and when adding to the formulation the quantity higher than 5% causes the increase of the precipitate, i.e. yield of the pharmaceutical composition is reduced and does not effect the quality of the composition in the direction of improvement. It should be marked that ammonia synthesis catalyst waste residues in complex with the other components provide the pharmaceutical composition having effective disinfecting property.

With the purpose of obtaining the pharmaceutical composition, using in the formulation of non-spent ammonia synthesis catalyst waste residues results in obtaining the pharmaceutical composition with less effective disinfecting property and causes rise in price of product.

Adding to the formulation of the citric acid the quantity lower than 0.1% does not increase the efficiency of action, and at adding higher than 2.0% changes sharply the pH of the solution.

Organic acid—citric acid added to the formulation represents tribasic oxyacid. Use of monobasic and dibasic organic acid is not permissible, as at interaction with the nitric acid they form highly inflammable and explosive substances.

Adding to the formulation of ethyl alcohol less than 0.1% is insufficient for the solvent function, and adding the quantity more than 5.0% increases insignificantly the efficiency of the disinfecting properties of the composition, and, at the same time, increases expenses.

For obtaining of the pharmaceutical composition to the formulation is added monoatomic alcohol—ethyl alcohol. Using in the formulation of other representatives of monoatomic alcohol or diatomic and triatomic alcohols enables to receive the pharmaceutical composition with the same technical effect. But these alcohols are poisonous and explosive substances and their use for obtaining of the formulation does not result in the safe pharmaceutical composition.

Adding to the formulation of aromatizer in the quantity of 0.05-0.1% does not effect the properties of the pharmaceutical composition, but at the same time gives a pleasant smell.

At obtaining of the homogeneous formulation, the ordinary water is used as a solvent.

In the method of obtaining the pharmaceutical composition during the interaction of reagents the process of esterification takes place according to the below presented flow diagram. At the same time, the acid loses hydroxyl group, alcohol—hydrogen. Both processes of esterification are invertible.

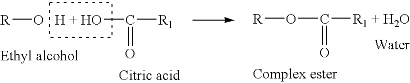

Ethyl alcohol is in reaction with the citric acid and in result, water and complex ester is received.

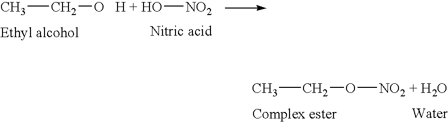

Ethyl alcohol with nitric acid also forms complex ester and water.

Complex esters are easily hydrolized. In water they decay into the initial components. Due to invertible character of esterification finally chemical equilibrium takes place.

In a reactor an isochoric process takes place. During the process the temperature and the pressure of water and gas are regulated automatically, and their ratio is a constant value const.

In the reactor the formulation for obtaining of the pharmaceutical composition is placed in the quantity of 70% of its volume. At such process the raw and power are consumed more rationally in the reactor.

At obtaining the pharmaceutical composition in the open system at room temperature and atmospheric pressure the time of performing the technological process increases and the obtained pharmaceutical composition is characterized with the low stability.

Initial Substances.

Ammonia synthesis catalyst waste residues—solid substance, stability of which at storing in waterproof iron boxes makes tens of years.

Nitric acid—in the concentration of 57% is a liquid that may be stored in black waterproof vessel for tens of years.

Citric acid (food)—colourless crystals or white powder without lumps, stability of which at storing in waterproof closed packs of “food” polyethylene films makes tens of years.

Ethyl alcohol—in the concentration of 96% is a colourless transparent liquid with a characteristic smell and poignant taste. It is stored in waterproof vessels. The shelf life of the alcohol is not limited.

Aromatizer—in the form of essence—concentrated aqueous alcoholic solution of essential oils. It represents homogenous transparent liquid characterized for certain plants odour and colour, mainly—lemon, mandarin, mint, rose, etc., stability of which at storing in the waterproof closed vessels makes tens of years.

The concrete examples of realization of the method for one liter of formulation to obtain pharmaceutical composition.

### EXAMPLE 1

In the reactor is placed 0.1 g of the ammonia synthesis catalyst waste milled residues comprising Fe₂O₃-57%, Cr₂O₇-15%, graphite—28%, 0.1 g of the citric acid and are mixed with 0.1 ml of 96% ethyl alcohol till obtaining a homogeneous mass. After to the mixture is added 3.0 ml of 57% nitric acid. The mixture is diluted with water up to one liter and is again mixed. Further the mixture is heated up to 75° C. and kept under pressure 7.0.10⁴ Pa till obtaining a transparent fraction. The transparent fraction is poured into a separate vessel and purified from admixtures, after 0.1 ml of aromatizer—lemon essence is added.

The obtained pharmaceutical composition is a 0.3% concentrate. The yield makes 980 ml. The wastes—solid ammonia synthesis catalyst waste residues˜≈10 g, which are washed with water, dried and used again in the formulation for obtaining the pharmaceutical composition.

During six hours an action of the obtained pharmaceutical composition on the physiological solution saturated with bacteria was effected. 100% bactericidal and virucidal activity was achieved within 20 minutes.

### EXAMPLE 2

In the reactor is placed 1.0 g of ammonia synthesis catalyst waste milled residues comprising Fe₂O₃-57%, Cr₂O₇-15%, graphite—28%, 0,8 g of the citric acid and are mixed with 1.0 ml of 96% ethyl alcohol till obtaining a homogeneous mass. After to the mixture is added 15.0 ml of 57% nitric acid. The mixture is diluted with water up to one liter and is again mixed. Further the mixture is heated up to 55° C. and kept under pressure 6.0.10⁴ Pa till obtaining of a transparent fraction. The transparent fraction is poured in a separate vessel and purified from admixtures, after 10 ml of aromatizer—lemon essence is added.

The obtained pharmaceutical composition is a 1.5% concentrate. The yield makes 975 ml. The wastes—solid ammonia synthesis catalyst waste residues—≈11 g, which are washed with water, dried and used again in the formulation for obtaining the pharmaceutical composition.

During six hours an action of the obtained pharmaceutical composition on the physiological solution saturated with bacteria was effected. 100% bactericidal effect was achieved within 5 minutes, and virucidal activity—within 20 minutes.

### EXAMPLE 3

In the reactor is placed 1.5 g of the ammonia synthesis catalyst waste milled residues comprising Fe₂O₃-57%, Cr₂O₇-15%, graphite—28%, 1.5 g of citric acid and are mixed with 2.5 ml of 96% ethyl alcohol till obtaining a homogeneous mass. After to the mixture is added 74 ml of 57% nitric acid. The mixture is diluted with water up to one liter and is again mixed. Further the mixture is heated up to 45° C. and kept under pressure 5.0.10⁴ Pa till obtaining of a transparent fraction. The transparent fraction is poured into a separate vessel and purified from admixtures, after 0.1 ml of aromatizer—lemon essence is added.

The obtained pharmaceutical composition is a 5% concentrate. The yield makes 970 ml. The wastes—solid ammonia synthesis catalyst waste residues makes—≈12 g, which are washed with water, dried and used again in the formulation for obtaining the pharmaceutical composition.

During six hours an action of the obtained pharmaceutical composition on the physiological solution saturated with bacteria was effected. 100% bactericidal effect was achieved within 5 minutes, and virucidal activity—within 10 minutes.

### EXAMPLE 4

In the reactor is placed 5 g of the ammonia synthesis catalyst waste milled residues comprising Fe₂O₃-57%, Cr₂O₇-15%, graphite—28%, 2 g of citric acid and are mixed with 5 ml of 96% ethyl alcohol till obtaining a homogeneous mass. After to the mixture is added 148 ml of 57% nitric acid. The mixture is diluted with water up to one liter and is again mixed. Further the mixture is heated up to 35° C and kept under pressure 4.0.10⁴ Pa till obtaining of a transparent fraction. The transparent fraction is poured into a separate vessel and purified from admixtures, after 0.1 ml of aromatizer—lemon essence is added.

The obtained pharmaceutical composition is a 10% concentrate. The yield makes 960 ml. The wastes—solid ammonia synthesis catalyst waste residues—≈14 g, which are washed with water, dried and used again in the formulation for obtaining the pharmaceutical composition.

During six hours an action of the obtained pharmaceutical composition on the physiological solution saturated with bacteria was effected. 100% bactericidal and virucidal activity was achieved within 5 minutes.

According to the method of Example 4 a pharmaceutical composition with the concentration 9.0-10,5% may be obtained, at the same time, it is characterized with the following physical and chemical and bacteriological indices:

Appearance—transparent liquid.

Smell—characteristic for alcohol and applied aromatizer.

pH—1.0-2,5

Density—1.050-1.060

Concentration—9.0÷10.5

Time of elimination of staphylococcus aurus, in minutes—not more than 30 (with 1% aqueous solution of the pharmaceutical composition).

Shelf life of the pharmaceutical composition is 2 years from the day of production. Is stored at the 5-35° C. temperature. Transportation is carried out at the temperature −5÷+35° C.

Chemical analysis of 9.0÷10.5 concentrate of the pharmaceutical composition is as follows:

C—organic—5.0 g/l; C—inorganic—0.3 g/l; Fe—0.5÷7 g/l; K—0.1÷0.7 g/l; Ca÷1÷6 g/l; Cu—1÷3 g/l; Al—3÷4 g/l; Si—0.3÷5 g/l; Cr—0.01÷0.5 g/l; Mg—0.1÷1.0 g/l; Mn—0.05÷2.0 g/l, etc., for example, Ni; H₂; NO; NO₂.

Sanitary and hygienic research of 10% pharmaceutical composition was performed for the following indices:

1. Irritant Action on Mucous Membrane of Eyes.

Result:—observation during 2 weeks revealed a slight irritation of the mucous membrane of eyes, epiphora. 2. Inhalation Effect.

Result:

1) During 60 minutes from the start of action in animals flaccidity and heavy breathing were observed. The downfall of animals was absent. Condition of animals was normalized after ending of the action.

2) During 30 minutes from beginning of action in animals light flaccidity and heavy breathing were observed. The downfall of animals was absent. Condition of animals was normalized after ending of the action.

Thus, index of sanitary and hygienic research equals to DL₅₀>5000.

At the same time, this index in below mentioned means is:

1. LISOFORMIN—3000 equals to DL₅₀=3000

2. Shulke & Mayr—DL₅₀=2000:3000

3. Septodor—DL₅₀=450

The obtained according to the method of example 4 pharmaceutical composition is a liquid 10% concentrate and is used as a reagent in the concentrations sufficient for elimination of microorganisms, but safe for medical personnel. It does not show corrosiveness in respect to the non-corrosive metals, glass, rubber and polymer materials.

The pharmaceutical composition is used for disinfecting, sterilization and antisepics in the form of 0.025-3% aqueous pharmaceutical composition. Preparation of the working solutions is performed by dilution with water.

At the same time, for preparation of 1 liter of aqueous solution of the pharmaceutical composition with the concentration 0.25% is taken 25 ml 10% pharmaceutical composition.

For 0.5% is taken 50 ml;

for 1.0% is taken 100 ml;

for 1.5% is taken 150 ml;

for 2.0% is taken 200 ml;

for 2.5% is taken 250 ml;

for 3.0% is taken 300 ml.

It is preferable to use these solutions during 20-30 days.

Before using these solutions it is necessary to clean preliminarily the work surface from different organic and mechanical contaminations. The traces of organic contaminations (for example, blood, fats, proteins, hydrocarbons, etc.) should not be removed from the work surface, as the disinfectant easily removes them from the surface, as well as performs their binding and solidification. Whereas, the existing analogues dissolve them contaminating the working solution making analogue disinfecting solutions useless for further use.

The proposed means may be drained from the solid admixtures by simple laboratory methods and reused.

Use of the proposed composition for disinfecting, sterilization and antiseptics of different work surfaces depends on the concentration of the solution and exposure time and causes bacteriostatic (prevents the growth and propagation of microbes), or bactericidal action (eliminates microbes) on the following kinds of bacteria and viruses (see Tables 1, 2 and 5). As seen from the Tables: (+) means the growth of bacteria and viruses; (−) means that growth was not observed.

Following conditions influence anti-microbe and anti-virus effect of the proposed composition:

Concentration of disinfecting solution and degree of its dissociation. The higher the concentration, the higher is the effect;

Duration of exposure time. By increasing the exposure time the efficiency increases and visa versa;
Degree of contamination with the high-molecular organic substances existing on the work surface. At disinfecting the surfaces contaminated with the organic substances the anti-microbe and anti-virus effect reduces and visa versa increases at reducing the degree of the contamination;
The quantity of viruses and bacteria in the medium of disinfectant action. By increasing the concentration of disinfectant means its anti-microbe and anti-virus effect increases;

Temperature of the disinfectant and working surface. At the time of treatment a slight increase or reduce of temperature takes place. But at the same time, the temperature affects the exposure time onto the working surface;

Kinds of microbes and viruses. The force of effect of disinfectant of the same concentration on the various types of microbes and viruses is not the same.

Aqueous solutions of the pharmaceutical composition are used for treatment of the public places—schools, kindergartens, markets, catering objects, cinemas, furniture, sanitary and technical equipment, table and laboratory glassware, for disinfecting and sterilization of medicinal premises; operation, antiseptic, reanimation medical-surgical wards, hospital appliances, such as linen, bandaging materials, as well as items from rubber, unstainable metals, plastics, etc., which are applied in medicinal and prophylactic institutions and for neutralization of patients' egesta and hospital infection.

Treatment with disinfecting, sterilizing and antiseptic solution is performed by means of full dipping in the solution, washing of walls and surfaces with a stream, by aerosol spraying, irrigation, using a rag saturated with the solution.

In tables 3, 4 and 6 the working solutions of the pharmaceutical composition and results of tests for disinfecting of working surfaces are presented. Notation conventions in the Table 4 are as follows:
(−)—not disinfected;
O—partially disinfected;
(+)—disinfected

Tests were performed in the following conditions:

Air humidity 75÷86%;

Solution temperature 20° C.;

Air temperature in the storehouse building 10° C.

As seen from Table 6, in examples 1, 2 the treatment of surfaces with 0.5; 1.0; 1.5; 2.0% aqueous solutions of the pharmaceutical composition is performed by exposition according to 30; 20; 10 and 5 minutes, by complete dipping into the solution or with a rag wetted in the solution for disinfection and sterilization of special danger bacteria, viruses, Staphylococcus aurus and hepatitis B.

In the example 3-1.0; 1.5; 2.0% aqueous solutions of the pharmaceutical composition are effective for disinfection of hospital infections by cleaning with a rag wetted in the solution or washing during 30; 10 and 5 minutes respectively.

In the example 4-1.0 and 2.0% aqueous solutions of the pharmaceutical composition reveal bactericidal, antivirus, fungicidal and anti-spore properties and are effective for disinfection and sterilization of medical appliances and apparatus, including medical appliances of non-rusting metal, platinum, glass, used in stomatology and surgery. Disinfection and sterilization is performed during 30 and 10 minutes respectively by complete dipping them into these solutions, providing thus the absence of air blebs and filling all the canals. Thickness of the solution layer above items should not be less than 1 cm.

In the examples 5 and 7-0.25; 0.5 and 1.0% aqueous solutions of the pharmaceutical composition are effective for disinfection and sterilization of cloths, hospital appliances, such as bed linen, smocks, bandaging material, buildings, furniture, etc. during 30; 15 and 5 minutes respectively by wetting the articles from fabric, and cleaning furniture with a rag wetted in the solution.

In the example 6-1.0 and 1.5% aqueous solutions of the pharmaceutical composition may be effectively used as skin antiseptic for sterilization of surgeons' and medical personnel hands, as well as operation and injection areas by treating hands and respective surfaces with the following drying. At the same time, within 3 and 1 minute they completely suppress bacteria, fungi, viruses.

During use the aqueous pharmaceutical composition is characterized with the following properties:

after drying it does not leave traces on the work surface;

it immediately begins active action at room temperature, if even a lowest concentration is used.

The proposed composition as an antimicrobial means according to the composition and mechanism of action refers to disinfecting and sterilizing means with acid properties.

Microbiological Activity

The preparation is efficient in respect to Gr(+), Gr(−) bacteria, mycobacterium of tuberculosis, mycelial fungus, yeast, viruses (pathogens of hepatitis B, C, D, HIV-infection, respiratory infections) and spores.

Chemical and biological essence of the pharmaceutical composition action may be explained as follows.

Acid medium at the local action on the cell of a living organism depending on concentration produces binding (at low concentration) or irritating, necrotizing action (at high concentrations). At the same time, interaction of acid with proteins takes place. The proteins lose water and coagulate forming albuminate.

At using mediums with increased acidity the formed albuminate is firm, that is why injury of tissue is a surface one (coagulation necrosis). This mechanism conditions also the antimicrobial action of acid mediums. At penetration in microbe cells they dissociate and cause (in microbe cells) binding of proteins and precipitation, in result bacteria either dies or further does not develop and propagate.

The proposed pharmaceutical composition has as well antiseptic properties.

As an antiseptic means, it is used for treatment of insignificant traumas of skin, such as scratches, fissures, cuts. At that 0.1-1% aqueous solution of the pharmaceutical composition is used. It is used by washing the injured place with the solution, or by a bandage wetted with the solution applied to wounds.

These procedures are repeated several times, as required.

Example of Using the Proposed Pharmaceutical Composition as an Antiseptic Means.

The pharmaceutical composition was tested by an emergency staff on 50 patients having subcutaneous wounds of various complexity on the body. In all the cases of treatment of the wounds the surface of the wound, as well as the skin around it, was cleaned and washed from contamination with the bandages treated with the 1% solution of the proposed pharmaceutical composition, after bandages treated in the same solution were applied. Such a treatment of the wound in all the cases was effective.

The proposed pharmaceutical composition, as an antiseptic means is characterized not only by good therapeutic action, but by simplicity of use.

Practical use has clearly shown that the proposed composition as an antiseptic is much more strong and efficient than boric acid and salicylic acid.

Use of the proposed invention enables to:

obtain a pharmaceutical composition for disinfection, sterilization and antiseptics, the use of which has a big economic effect, conditioned by the possibility of using the solution of low concentration during short time of exposition.

obtain a pharmaceutical composition that may be used as a reagent providing antimicrobial, viricidal, fungicidal, sporecidal, bactericidal and tuberculocudal action, which does not provide the damaging action onto the working surfaces, instruments, (including endoscopes and thermounstable materials), having washing properties, pleasant fresh odor, biological decomposition and not containing phenols, phosphates and oxidizing substances.

obtain a pharmaceutical composition by simple not requiring complex technological equipment method;

manufacture and use aqueous solutions of the pharmaceutical composition without ventilation and individual protection means as it does not have toxic and allergic action on medical personnel;

provide with long persistent antimicrobial action preventing propagation of microorganisms as a minimum during 2-4 hours after use;

maintain ecology of the environment by creating non-waste technology.

### Results of Testing the Bactericidal Action

TABLE 1

| | | Concentration of | | | | | | |
|---|---|---|---|---|---|---|---|---|
| | Exposition | disinfecting solution, % | | | | | | Con- |
| Name of bacterium | min | 0.05 | 0.1 | 0.2 | 0.5 | 1 | 2 | trol |
| Jersinia pertis | 3 | − | − | − | − | − | − | + |
| | 5 | − | − | − | − | − | − | + |
| Jersinia enterocolitica | 3 | + | + | + | − | − | − | + |
| | 5 | + | + | − | − | − | − | + |
| Salm. typhimurium | 3 | + | + | + | − | − | − | + |
| | 5 | + | + | − | − | − | − | + |
| Klebsiela aerobacter | 3 | + | + | + | − | − | − | + |
| | 5 | + | + | − | − | − | − | + |
| Vibrio cholerac | 3 | + | − | − | − | − | − | + |
| | 5 | − | − | − | − | − | − | + |
| Shigella disentericue | 3 | + | − | − | − | − | − | + |
| | 5 | − | − | − | − | − | − | + |
| Escherichia coli | 3 | + | + | + | + | − | − | + |
| | 5 | + | + | + | − | − | − | + |
| Staphilococcus aurus | 3 | + | + | + | + | − | − | + |
| | 5 | + | + | + | + | − | − | + |

### Results of Testing the Virusicidal Action

TABLE 2

| | | Concentration of | | | | | | |
|---|---|---|---|---|---|---|---|---|
| | Exposition | disinfecting solution, % | | | | | | |
| Name of bacteria | min | 0.05 | 0.1 | 0.2 | 0.5 | 1 | 2 | Control |
| The so-called | 3 | − | − | − | − | − | − | + |
| West-Nilus Fever | 5 | − | − | − | − | − | − | + |
| Influenza virus | 3 | + | − | − | − | − | − | + |
| A-1 | 5 | − | − | − | − | − | − | + |
| Coxsackie virus | 3 | + | + | − | − | − | − | + |
| B-1 | 5 | + | + | − | − | − | − | + |
| Hepatitis virus | 3 | + | + | + | + | − | − | + |
| A-1 | 5 | + | + | + | + | − | − | + |

Notation conventions:
(+) growth observed;
(−) growth not observed

### Sphere of Application and Time of Treatment, Taking into Account Concentrations

TABLE 3

| | Concentration, % | | | |
|---|---|---|---|---|
| Name of appointment | 0.5% | 1.0% | 1.5% | 2.0% |
| Disinfecting of bacteria | | 30 minutes | | 10 minutes |
| and viruses | | | | |
| Staphylococcus aurus | | | 60 min. | 30 minutes |
| and hepatitis B | | | | |
| Hospital infection | | | | 60 min. |
| Treatment of tools and | | | | 30 minutes |
| surfaces | | | | |
| Treatment of tissues | 60 min. | | | |
| Treatment of hands | | 3 minutes | | |

### Results of Surfaces Treatment

TABLE 4

| | | | Number of the | | | | |
|---|---|---|---|---|---|---|---|
| | | Solution | executed treatments | | | | |
| Type of | Concentration | expenditure | Number of | | Number of | Disinfecting | Exposition time |
| bacteria | of the solution % | l/m² | times | l/m² | Experiments | results | in hours |
| Staphylococcus | 0.25 | 0.5 | 2 | (0.250) | 3 | + | 3 |
| aurus | 0.5 | 0.5 | 1 | (0.5) | 3 | ο | 3 |
| | 0.5 | 0.4 | 2 | (0.2) | 3 | − | 2 |
| | 1.0 | 0.2 | 1 | (0.2) | 3 | + | 3 |
| | 1.0 | 0.3 | 2 | (0.15) | 3 | − | 3 |

### Results of Testing the Bactericidal Action

TABLE 5

| | | Concentration of | | | | | | |
|---|---|---|---|---|---|---|---|---|
| | Exposition | disinfectant solution % | | | | | | |
| Name of microorganisms | Time in min | 0.025 | 0.05 | 0.1 | 0.25 | 0.5 | 1 | Control |
| P. aeruginozae | 5 | ++ | +− | −− | −− | −− | −− | ++ |
| | 15 | +− | +− | −− | −− | −− | −− | |
| | 30 | −− | −− | −− | −− | −− | −− | |
| | 60 | −− | −− | −− | | | | |
| Escherichia coli | 5 | ++ | ++ | ++ | ++ | −− | −− | ++ |
| | 15 | +− | −− | −− | −− | −− | −− | |
| | 30 | +− | −− | −− | −− | −− | −− | |
| | 60 | −− | −− | −− | | | | |
| Shigella dysenteriae | 5 | ++ | ++ | ++ | −− | −− | −− | ++ |
| | 15 | ++ | ++ | ++ | −− | −− | −− | |
| | 30 | ++ | +− | +− | −− | −− | −− | |
| | 60 | ++ | −− | −− | | | | |
| Staphilococcus aureus | 5 | ++ | ++ | ++ | +− | −− | −− | ++ |
| | 15 | ++ | ++ | ++ | −− | −− | −− | |
| | 30 | ++ | ++ | −− | −− | −− | −− | |
| | 60 | ++ | ++ | −− | | | | |
| S. typhimurium | 5 | ++ | ++ | −− | −− | −− | −− | ++ |
| | 15 | ++ | −− | −− | −− | −− | −− | |
| | 30 | −− | −− | −− | −− | −− | −− | |
| | 60 | −− | −− | −− | | | | |
| B. cereus | 5 | ++ | ++ | ++ | ++ | ++ | ++ | ++ |
| | 15 | ++ | ++ | ++ | ++ | +− | −− | |
| | 30 | ++ | ++ | ++ | +− | −− | −− | |
| | 60 | ++ | +− | −− | | | | |
| Klebsiella aerobacter | 5 | ++ | ++ | ++ | ++ | −− | −− | ++ |
| | 15 | ++ | ++ | +− | −− | −− | −− | |
| | 30 | ++ | ++ | +− | −− | −− | −− | |
| | 60 | +− | +− | −− | | | | |

Notation conventions:
(+) growth observed;
(−) growth not observed

### Field of Application and Time of Treatment

### Depending on Concentration of the Pharamaceutical Composition

TABLE 6
Field of Application and Time of Treatment
Depending on Concentration of the Pharamaceutical Composition

| No of | | Concentration % | | | | | Method of |
|---|---|---|---|---|---|---|---|
| Example | Object of Treatment | 0.25 | 0.5 | 1.0 | 1.5 | 2.0 | Treatment |
| 1 | Disinfection of | | 30 min. | 20 min. | 10 min. | 5 min. | Method of dipping |
| | bacteria, viruses and | | | | | | or cleaning with |
| | spores | | | | | | cloth wetted in the |
| | | | | | | | solution |
| 2 | Staphilococcus aureus | | | 30 min. | 20 min. | 10 min. | Method of dipping |
| | and hepatitis B | | | | | | or cleaning with |
| | | | | | | | cloth wetted in the |
| | | | | | | | solution |
| 3 | Hospital infections | | | 30 min. | 10 min. | 5 min. | Washing or |
| | | | | | | | cleaning with |
| | | | | | | | wetted cloth |
| 4 | Treatment of | | | 30 min. | | 10 min. | Dipping in the |
| | appliances and | | | | | | solution |
| | surfaces | | | | | | |
| 5 | Treatment of cloths | 30 min. | 15 min. | | | | Soaking in the |
| | | | | | | | solution |
| 6 | Hygienic treatment of | | | 3 min. | 1 min. | | Treatment of hands |
| | hands | | | | | | with the solution |
| | | | | | | | with following |
| | | | | | | | drying |
| 7 | Treatment of hospitals, | 30 min. | 15 min. | 5 min. | | | Soaking of linen in |
| | surgical garb, coats, | | | | | | the solution, |
| | bed linen, etc. | | | | | | cleaning or |
| | | | | | | | washing with the |
| | | | | | | | wet cloth |

1. A formulation for obtaining a pharmaceutical composition comprising acid, said acid comprises a weak nitric acid; ammonia synthesis catalyst waste residues, citric acid, ethyl alcohol, water and aromatizer in the ratio of components, in weight %:

| | |
|---|---|
| weak nitric acid | 0.3-10.0; |
| ammonia synthesis catalyst waste residues | 0.1-5.0; |
| citric acid | 0.1-2.0; |
| ethyl alcohol | 0.1-5.0; |
| aromatizer | 0.05-0.1; and |
| water | the rest. |

2. The formulation according to claim 1, wherein the ammonia synthesis catalyst waste residues comprises an oxide of iron, an oxide of chromium and graphite.

3. The formulation according to claim 2, wherein the ammonia synthesis catalyst waste residues comprise not less than 50% by weight of iron oxide.

4. A method for preparing a pharmaceutical composition comprising the steps of:
a) mixing ammonia synthesis catalyst waste residues and citric acid with ethyl alcohol to obtain a homogeneous mass;
b) adding to the mass weak nitric acid to obtain a mixture;
c) diluting the mixture obtained in step b) with water;
d) mixing until a transparent fraction is obtained;
e) purifying the transparent fraction;
f) adding aromatizer, to obtain the composition comprising in weight %

| | |
|---|---|
| weak nitric acid | 0.3-10.0; |
| ammonia synthesis catalyst waste residues | 0.1-5.0; |
| citric acid | 0.1-2.0; |
| ethyl alcohol | 0.1-5.0; |
| aromatizer | 0.05-0.1; and |
| water | the rest. |

5. The method according to claim 4, wherein the method is carried out in a closed system.

6. The method according to claim 5, wherein the closed system is a reactor.

7. The method according to claim 6, wherein the process is carried out in a reactor at a temperature of 25-90° C. and under pressure of 1.013.10⁴-1.013.10⁵ Pa.

8. A pharmaceutical composition obtained according to the method of claim 4.

9. A pharmaceutical composition obtained according to the method of claim 7.

10. A method of disinfecting, sterilization and antiseptic application of a surface, comprising treatment of the surface with the pharmaceutical composition, according to claim 1.

11. The method according to claim 10, wherein the pharmaceutical composition is aqueous and is applied to the surface after dilution with water preferably in concentration 0.025-3.0%.

12. The method according to claim 10, wherein the pharmaceutical composition is diluted to a concentration 0.025-3% v/v.

13. The method according to claim 10, wherein the treatment of the surface is performed by a method selected from the group consisting of a dipping up to complete covering by the solution, washing of walls and surfaces with a stream, by aerosol spraying, irrigation, cleaning of surfaces with a cloth wetted in solution, washing, or applying a bandage wetted with the composition.
